(19) United States
(12) Patent Application Publication
(10) Pub. No.: US 2008/0139988 A1
(43) Pub. Date: Jun. 12, 2008

(54) BANDAGE, TOPICAL AGENT-BEARING PACKAGE AND TOPICAL AGENT-BEARING ELEMENT

(75) Inventors: Tamir Dayan, Tel Aviv (IL); Oz Drimer, Herzliya (IL)

Correspondence Address: KREMBLAS, FOSTER, PHILLIPS & POLLICK, 7632 SLATE RIDGE BOULEVARD, REYNOLDSBURG, OH, 43068, US

(73) Assignee: PERSYS MEDICAL LTD., Herzliya (IL)

(21) Appl. No.: 11/949,180

(22) Filed: Dec. 3, 2007

Related U.S. Application Data
(60) Provisional application No. 60/873,271, filed on Dec. 7, 2006.
(60) Provisional application No. 60/898,417, filed on Jan. 31, 2007.

(51) Int. Cl. A61F 13/00

(52) U.S. Cl. 602/48

(57) ABSTRACT

An improved bandage including a strip of fabric for winding about a body portion having a wound; a dressing, formed integrally with said strip of fabric, for placement over the wound; and a pocket formed over said dressing, adapted to contain therein a topical agent, preferably a hemostatic agent, for treating the wound. Also envisioned is a topical agent-bearing package which comprises a plurality of integrally formed discrete elements, each formed of a permeable exterior layer containing a volume of a topical agent and an intervening, weakened region formed between each of said discrete elements, adapted to facilitate separation of at least one of said discrete elements from the remainder of said plurality of discrete elements.

### REFERENCE TO RELATED APPLICATIONS

This application claims priority to U.S. Provisional Application No. 60/873,271 filed 7 Dec. 2006, and U.S. Provisional Application No. 60/898,417 filed 31 Jan. 2007, the entire contents of each of which are hereby incorporated by reference.

### FIELD OF THE INVENTION

The present invention relates to the treatment of open wounds in the field.

### BACKGROUND OF THE INVENTION

Different types of bandages are known in the art, useful for binding injured limbs or other body parts. Among common types of bandages are those made from a crepe-type fabric, as well as sports bandages, elastic bandages, those made to include wound dressings and medications, as well as many others. Most bandages are formed in a roll, so as to be convenient to hold and maneuver in one hand when being used to bind a wounded or injured limb. This is particularly convenient as the bandage may be several meters in length, and must be kept clean and sterile prior to use. This is especially true when using bandages as field dressings, such as in combat zones, where open wounds must be quickly and efficiently bandaged prior to evacuating the patient to a clinic or hospital.

The use of hemostats for the control of bleeding, both in surgery and in emergency medicine, is also known, and normally form part of the first aid equipment carried by paramedical personnel.

While one type of a hemostat is a clamp, used to apply pressure to the wound so as to stop the bleeding, another type of hemostat is a topical agent composed of resorbable microfibrillar collagen. As described at www.answers.com, the microfibrillar collagen hemostat, known also by its acronym MCH, attracts platelets and allows for the formation of a blood clot when it comes into contact with blood. This material is pressed against a bleeding site and the collagen attracts and helps with the clotting process to eventually stop bleeding.

Various products are sold which incorporate hemostats. These include INSTAT™ and INSTAT MCH™ distributed by Johnson & Johnson, as well as QuikClot 1st Response™, manufactured by Z-Medica Corporation of 4 Fairfield Blvd., Wallingford, Conn. 06492 USA. The QuikClot 1st Response™ product is contained in a self-contained sponge which may be inserted into a wound in order to rapidly stop the bleeding. Additional products include the hemostatsic material CELOX™ manufactured by SAM Medical Products of 4909 S Coast Hwy, Step 245, Newport, Oreg. 97364, USA; and the HEMCON® Hemorrhage Control Bandages and CHITOFLEX™ dressing, manufactured by HemCon Medical Technologies, Inc. 10575 SW Cascade Avenue, Suite 130, Portland, Oreg. 97223-4363 USA.

### SUMMARY OF THE INVENTION

The present invention seeks to provide improved means of applying topical medical agents to open wounds, and particularly to bleeding wounds.

There is thus provided, in accordance with a preferred embodiment of the present invention, an improved bandage which includes a strip of fabric for winding about a body portion having a wound; a dressing, formed integrally with the strip of fabric, for placement over the wound; and a pocket formed over the dressing, adapted to contain therein a topical agent for treating the wound.

Additionally in accordance with the present invention, the pocket is arranged so as to be brought into contact with a wound being dressed, and is permeable to the passage of fluids therethrough.

Further in accordance with the present invention, the pocket is formed with a plurality of openings, thereby to permit body fluids emanating from the wound to contact the contents of the pocket.

Additionally in accordance with the present invention, the pocket is formed of a fluid permeable material thereby to permit body fluids emanating from the wound to contact the contents of the pocket.

Further in accordance with the present invention, the pocket is formed of a fluid permeable material having formed therein a plurality of openings, thereby to permit body fluids emanating from the wound to contact the contents of the pocket.

Additionally in accordance with the present invention, the topical agent is provided as a discrete, removable topical agent-bearing unit.

Further in accordance with the present invention, the topical agent is a hemostatic agent, and the topical agent-bearing unit is a discrete unit containing the hemostatic agent.

In accordance with an alternative embodiment of the present invention, there is provided a hemostatic bandage which includes a strip of fabric for winding about a body portion having a bleeding wound; a dressing, formed integrally with the strip of fabric, for placement over the wound; a pocket formed over the dressing; and a hemostatic agent contained within the pocket for stopping blood flow from the wound.

Additionally in accordance with the present embodiment, the pocket is arranged so as to be brought into contact with a wound being dressed, and is permeable to the passage of blood therethrough, in active contact with the hemostatic agent.

Further in accordance with the present embodiment, the pocket is formed with a plurality of openings.

Additionally in accordance with the present embodiment, the pocket is formed of a fluid permeable material.

Further in accordance with the present embodiment, the pocket is formed of a fluid permeable material having formed therein a plurality of openings.

Additionally in accordance with the present embodiment, the hemostatic agent is provided as a discrete, removable hemostatic agent-bearing unit.

In accordance with another alternative embodiment of the present invention, there is provided a topical agent-bearing package which includes:

a plurality of integrally formed discrete elements, each formed of a permeable exterior layer containing a volume of a topical agent; and

an intervening, weakened region formed between each of the discrete elements, adapted to facilitate separation of one or more of the discrete elements from the remainder of the plurality of discrete elements.

Additionally in accordance with the present embodiment, each discrete element has an elongate, finger-like shape.

Further in accordance with the present embodiment, each intervening region is perforated.

Additionally in accordance with the present embodiment, each discrete element has a tab for assisting in grasping the element and thus in removal of the element from the remainder of the plurality of discrete elements.

Further in accordance with the present embodiment, the topical agent is a hemostatic agent.

Additionally in accordance with the present embodiment, the topical agent-bearing package is adapted for use with the above-described improved bandage.

Further in accordance with the present embodiment, the topical agent-bearing package is adapted for use with the above-described hemostatic bandage.

In accordance with yet a further embodiment of the invention, there is provided apparatus for stopping bleeding from a deep narrow wound, including an elongate hemostatic element formed of a permeable exterior layer containing a volume of a hemostatic agent, shaped for lengthwise insertion into the wound.

In accordance with yet a further embodiment of the present invention, there is provided an improved bandage which includes:

a strip of fabric for winding about a body portion having a wound;

a dressing, formed integrally with the strip of fabric, for placement over the wound; and

a plurality of elongate pockets formed over the dressing, each pocket adapted to contain a single, discrete, topical agent-bearing element formed of a permeable exterior layer containing a volume of a topical agent for treating a bleeding wound.

Preferably, the topical agent is a hemostatic agent.

### DETAILED DESCRIPTION OF THE PRESENT INVENTION

The present invention will be more fully understood and appreciated from the following detailed description, taken in conjunction with the drawings, in which:

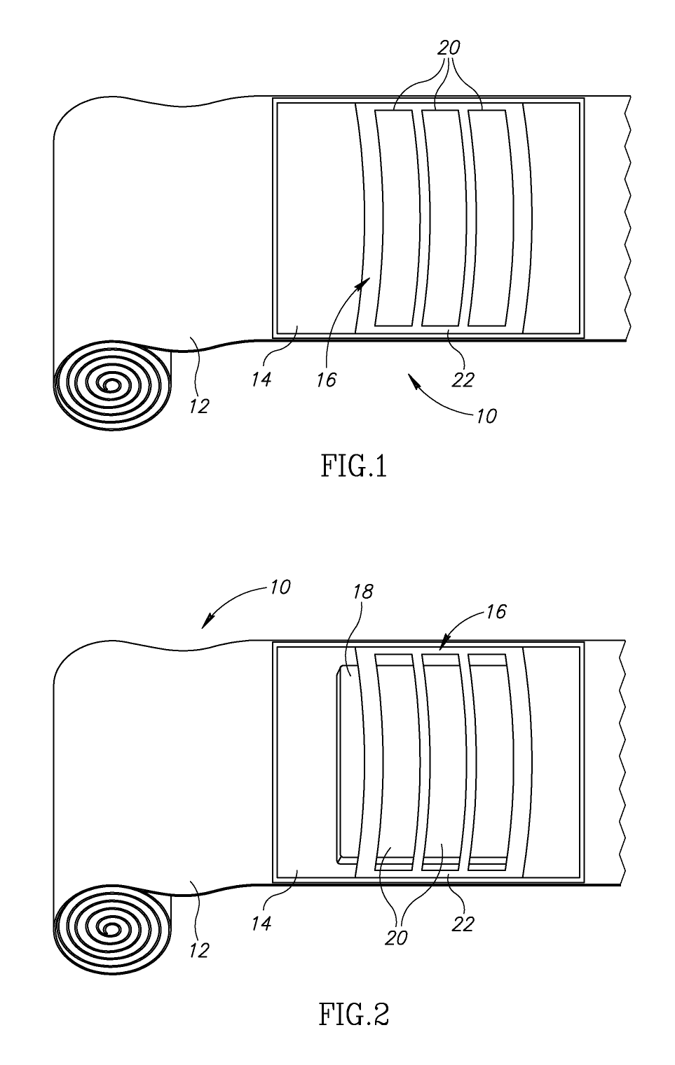

FIG. 1 is a pictorial representation of the bandage of the present invention;

FIG. 2 is a pictorial representation similar to that of FIG. 1, but having a topical agent-bearing unit contained in the bandage pocket;

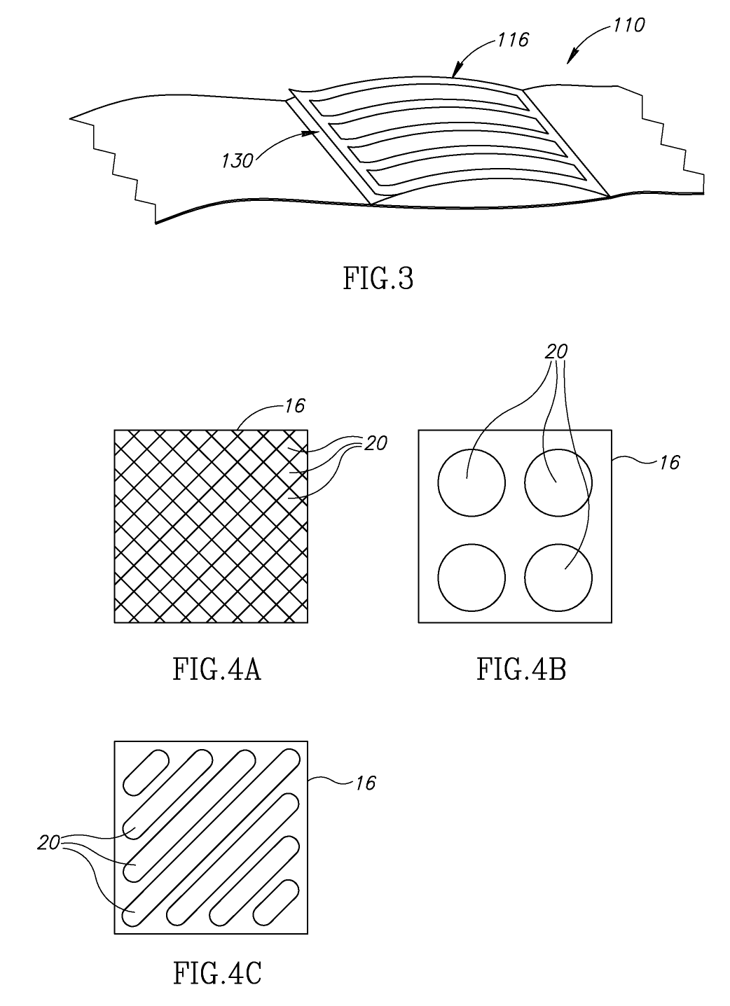

FIG. 3 is a pictorial representation showing a bandage of the invention, but having a pocket formed according to an alternative embodiment of the invention;

FIGS. 4A-4C are illustrations showing different types of pockets that may be used with the bandage of the present invention;

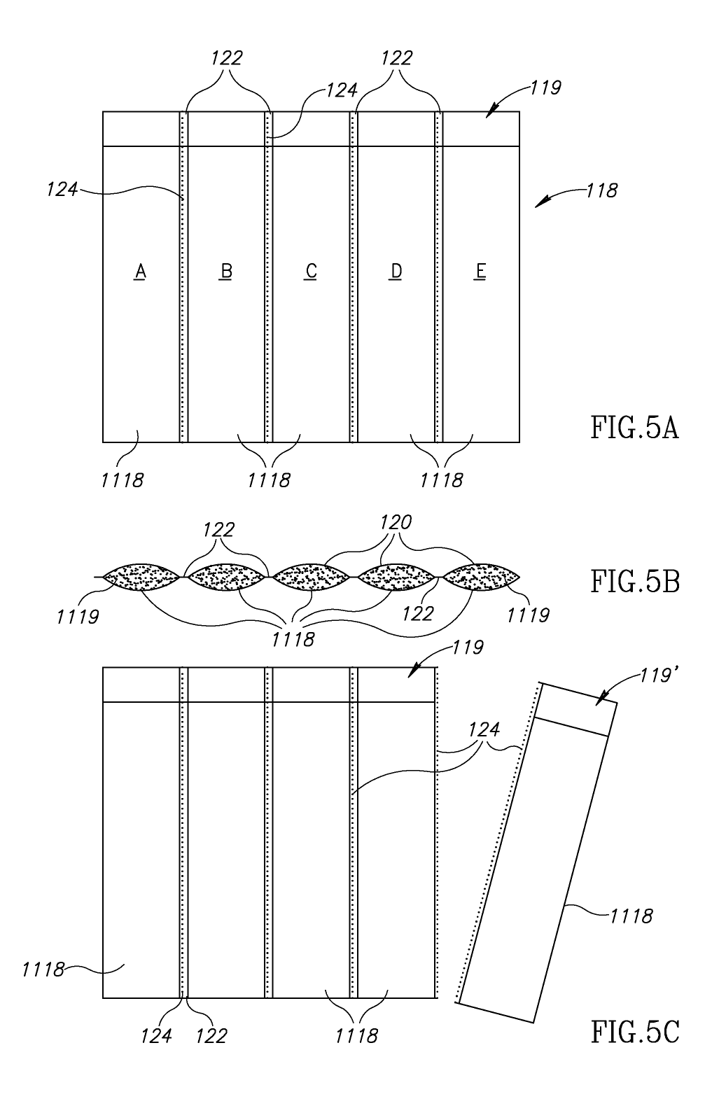

FIGS. 5A and 5B are schematic plan and end views, respectively, of a divisible topical agent-bearing package useful in conjunction with the bandage of FIGS. 1-4C;

FIG. 5C is a schematic plan view of the divisible topical agent-bearing package of FIGS. 5A and 5B, showing detachment of an element thereof;

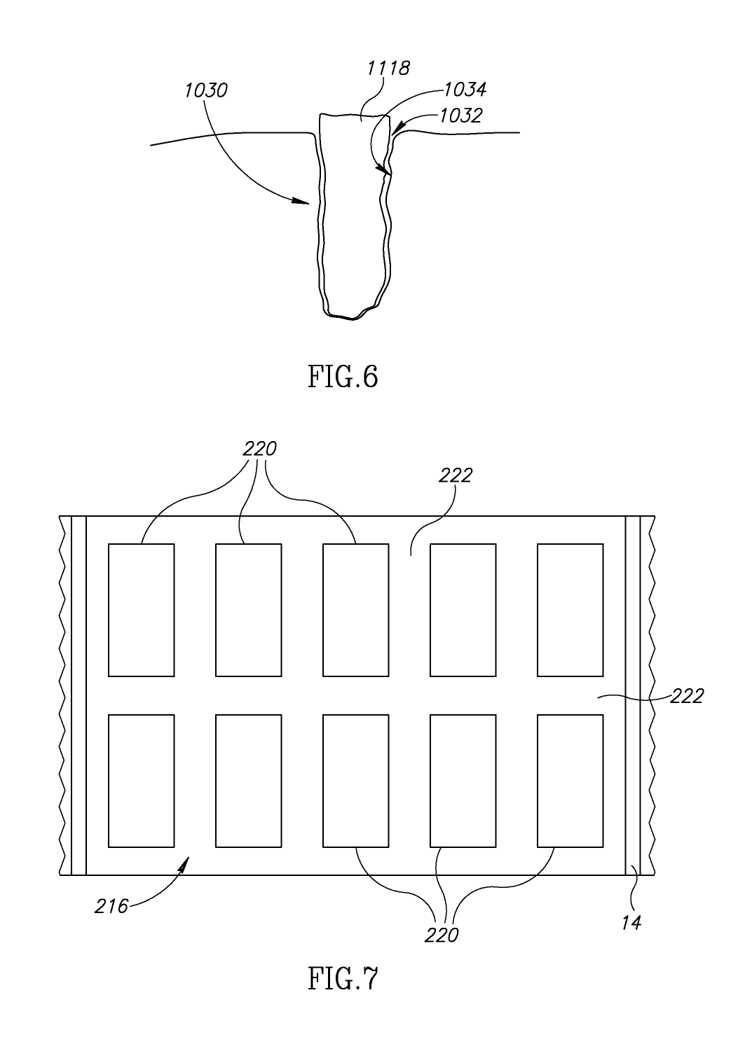

FIG. 6 is a schematic illustration showing use of a topical agent-bearing element of the invention, in treatment of a deep narrow wound;

FIG. 7 is a diagrammatic representation of a further variation of the bandage of FIGS. 1 and 2;

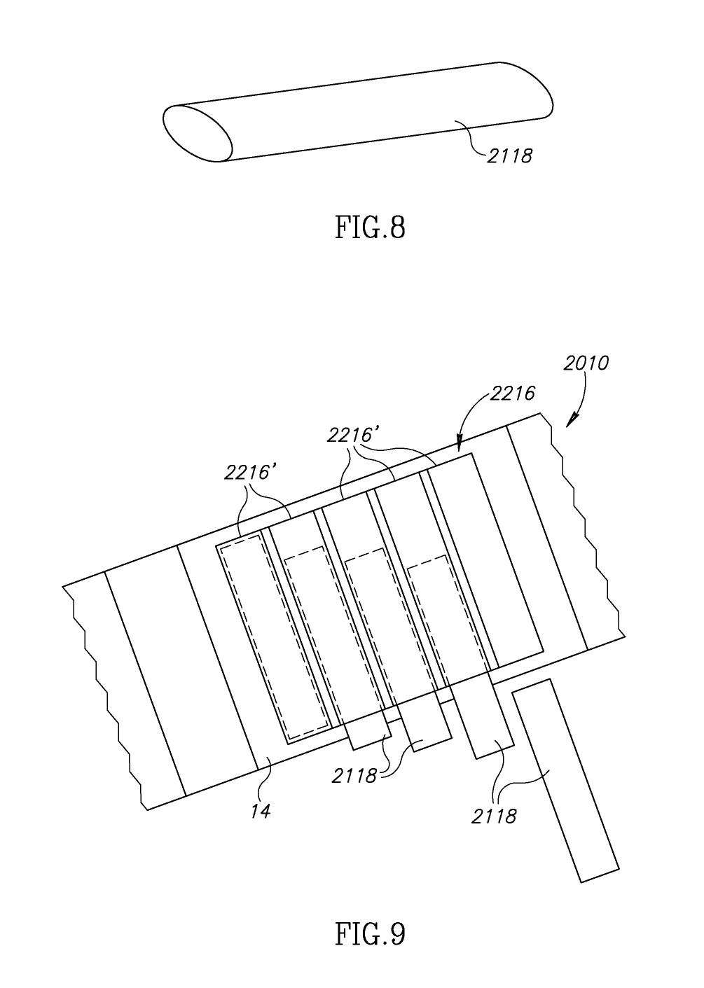

FIG. 8 is a schematic illustration of a discrete elongate topical agent-bearing element in accordance with an embodiment of the invention; and

FIG. 9 is a schematic illustration of a bandage having a plurality of pockets, each adapted to contain a single elongate topical agent-bearing element as shown in FIG. 8.

### DETAILED DESCRIPTION OF THE INVENTION

Referring now to FIGS. 1 and 2, there is provided an improved bandage, referenced generally 10, which is formed of a strip of fabric 12 for winding about a body portion having a wound; a dressing 14, formed integrally with the strip of fabric, for placement over the wound; and a pocket 16 formed over the dressing, adapted to contain therein a unit 18 (FIG. 2) of a topical agent for treating the wound. The topical agent may be any type of topical agent useful for treatment of an open wound. However, in accordance with a preferred embodiment of the present invention, the topical agent is a hemostatic agent, used for stopping bleeding, and may be such as found in a commercial product such as INSTAT™ or INSTAT MCH™ distributed by Johnson & Johnson; in a discrete, sponge-like unit 18 (FIG. 2), such as sold as QuikClot 1st Response™, manufactured by Z-Medica Corporation of 4 Fairfield Blvd., Wallingford, Conn. 06492 USA; in the hemostatsic material CELOX™ manufactured by SAM Medical Products of 4909 S Coast Hwy, Step 245, Newport, Oreg. 97364, USA; or in the HEMCON® Hemorrhage Control Bandages and CHITOFLEX™ dressing, manufactured by HemCon Medical Technologies, Inc. 10575 SW Cascade Avenue, Suite 130, Portland, Oreg. 97223-4363 USA.

As seen, the pocket 16 is fastened over the dressing 14, and is positioned so as to be brought into contact with a wound being dressed. In order to facilitate contact of the topical agent with the wound, the pocket 16 is formed so as to be permeable to the passage of fluids therethrough.

Pocket 16 may be formed so as to be permeable either by virtue of being formed from a permeable fabric 22, such as a suitable non-stick dressing; alternatively, it may have formed therein a plurality of cut-outs 20, through which the wound fluids may contact the topical agent. In accordance with a most preferred embodiment of the invention, however, the pocket is formed from a fluid-permeable fabric 22, and also has a plurality of cut-outs 20 formed therein.

Referring now to FIG. 2, the bandage 10 is shown with a topical agent unit 18 in place inside pocket 16. It is clear that unit 18 may be removed, if desired, although the bandage is used most advantageously in a situation in which the unit 18 is required, and all that is required of the paramedic is to bind the wound with the bandage without first having to apply the topical agent as a separate step. Optionally, if the topical agent is not required, it can be easily slipped out of the pocket 16.

Referring now briefly to FIG. 3, there is seen an additional bandage 110, which is similar to bandage 10 (FIGS. 1 and 2), but has a differently configured pocket 116. A topical agent unit (not shown) may be inserted into pocket 116 via an opening 130.

FIG. 4A shows a pocket 16 which is formed from a mesh-type of fabric, having a large number of relatively small cut-outs or openings 20.

FIGS. 4B and 4C are examples of pockets having differently configured cut-outs or openings 20. Clearly, any type of suitably configured openings can be provided, there being no particular importance to their shape or number, as long as they facilitate adequate fluid communication between the topical agent and fluids emanating from a wound to be bound and dressed.

Referring now to FIGS. 5A, 5B and 5C, there is seen a divisible topical agent-bearing package, referenced generally 118, useful in conjunction with any of the bandages of the invention.

In accordance with the present embodiment, package 118, which preferably contains a hemostat, substantially as described above, is sub-divided into a plurality of discrete, elongate, finger-like elements 1118. Five such elements are shown, although this is by way of example only. Each element 1118 is sealed and contains a predetermined quantity of hemostat, referenced 1119 (FIG. 5B). The elements 1118 are formed within a common outer fabric layer 120, and are separated by intervening regions 122 in the fabric which are weakened along separation lines 124, such as lines of perforation, so as to facilitate separation of the elements 1118 from each other, as by tearing. This is illustrated schematically in FIG. 5C.

Referring now also to FIG. 6, a particular advantage of the divisible package 118 of the present embodiment is that it provides an improved means of stopping bleeding from discrete wounds such as some bullet wounds. More specifically, in order to stop the bleeding from a deep wound 1030 having a relatively narrow opening 1032, binding the wound by use of a relatively wide pad containing a hemostat may not be very effective. However, in accordance with the present invention, one or more elements 1118 may be separated from the remainder of the package 118, and inserted lengthwise into the wound 1030, thereby contacting the inward-facing walls 1034 of the wound with a relatively large surface area of the hemostat-containing element 1118. Contacting the immediate area of the wound in this manner will clearly be more effective to stop bleeding quickly, than placement of a pad over the entrance to the wound.

Referring once more to FIGS. 5A and 5C, and in accordance with a preferred embodiment of the invention, package 118 has a wide fabric border strip 119 along one or both elongate edges (only one edge is illustrated), such that each discrete element 1118 has a tab 119′ which can be grasped between the fingers of a user, so as to assist in removal of the element 1118 from the remainder of said plurality of discrete elements without contaminating the material through which the topical agent and blood will contact each other.

Referring now briefly to FIG. 7, there is shown a further variation of the bandage of FIGS. 1 and 2. In the present embodiment, instead of the elongate cut-outs 20 of pocket 16 (FIGS. 1 and 2) which span substantially the full height of the pocket, pocket 216 has formed therein a plurality of smaller cut-outs 220, separated by a bridge portion 222, which serves to reinforce the pocket and further secure the topical agent-bearing unit therein. This is particularly useful when using the divisible package 118 of FIGS. 5A-5C, as the bridge portion 222 secures the remaining one or more elements 1118 of the package within pocket 216, after removal of one or more of the discrete elements 1118 from package 118.

Referring now to FIG. 8, there is shown a single, discrete, elongate topical agent-bearing element 2118. Element 2118, which typically is a hemostatic element, is generally similar to elements 1118 shown and described above in conjunction with FIGS. 5A-5C, except that it is totally self-contained, and, as such, may be used as described in conjunction with FIG. 6, without having to be torn off from the remainder of the package 118, as described above.

Referring now also to FIG. 9, there is shown a bandage 2010 which is a variation of the pocket-bearing bandage 10, shown and described hereinabove in conjunction with FIGS. 1 and 2. In the present embodiment there is provided a topical agent-bearing portion 2216 which has a plurality of pockets 2216′, each configured to contain a discrete, elongate topical agent-bearing element 2118, shown and described above in conjunction with FIG. 8. In the present illustration there are shown, by way of non-limiting example, five pockets, each with an element 2118 in a different state of insertion with respect to a selected pocket 2216′. Clearly, in a variation of this embodiment, there may be provided differently sized pockets and elements, each having a different sized surface area for differently sized wounds.

It will be appreciated by persons skilled in the art that the scope of the present invention is not limited by what has been shown and described hereinabove. Rather, the scope of the invention is limited solely by the claims, which follow.

1. An improved bandage which includes:
a strip of fabric for winding about a body portion having a wound;
a dressing, formed integrally with said strip of fabric, for placement over the wound; and
a pocket formed over said dressing, adapted to contain therein a topical agent for treating the wound.

2. An improved bandage according to claim 1, wherein said pocket is arranged so as to be brought into contact with a wound being dressed, and is permeable to the passage of fluid substances therethrough.

3. An improved bandage according to claim 2, wherein said pocket is formed with a plurality of openings, thereby to permit body fluids emanating from the wound to contact the contents of said pocket.

4. An improved bandage according to claim 2, wherein said pocket is formed of a fluid permeable material thereby to permit body fluids emanating from the wound to contact the contents of said pocket.

5. An improved bandage according to claim 2, wherein said pocket is formed of a fluid permeable material having formed therein a plurality of openings, thereby to permit body fluids emanating from the wound to contact the contents of said pocket.

6. An improved bandage according to claim 1, wherein said topical agent is provided as a discrete, removable topical agent-bearing unit.

7. An improved bandage according to claim 6, wherein said topical agent is a hemostatic agent, and said topical agent-bearing unit is a discrete unit containing said hemostatic agent.

8. A hemostatic bandage which includes:
a strip of fabric for winding about a body portion having a bleeding wound;
a dressing, formed integrally with said strip of fabric, for placement over the wound;
a pocket formed over said dressing; and
a hemostatic agent contained within said pocket for stopping blood flow from the wound.

9. A hemostatic bandage according to claim 8, wherein said pocket is arranged so as to be brought into contact with a wound being dressed, and is permeable to the passage of blood therethrough, in active contact with said hemostatic agent.

10. A hemostatic bandage according to claim 9, wherein said pocket is formed with a plurality of openings.

11. A hemostatic bandage according to claim 9, wherein said pocket is formed of a fluid permeable material.

12. A hemostatic bandage according to claim 9, wherein said pocket is formed of a fluid permeable material having formed therein a plurality of openings.

13. A hemostatic bandage according to claim 8, wherein said hemostatic agent is provided as a discrete, removable hemostatic agent-bearing unit.

14. A topical agent-bearing package which comprises:
a plurality of integrally formed discrete elements, each formed of a permeable exterior layer containing a volume of a topical agent; and
an intervening, weakened region formed between each of said discrete elements, adapted to facilitate separation of at least one of said discrete elements from the remainder of said plurality of discrete elements.

15. A package according to claim 14, wherein each said discrete element has an elongate, finger-like shape.

16. A package according to claim 14, wherein each said intervening region is perforated.

17. A package according to claim 16, wherein each said discrete element has a tab for assisting in grasping said element and thus in removal of said element from the remainder of said plurality of discrete elements.

18. A package according to claim 14, wherein said topical agent is a hemostatic agent.

19. A topical agent-bearing package according to claim 14, adapted for use with said improved bandage of claim 1.

20. A topical agent-bearing package according to claim 14, adapted for use with said hemostatic bandage of claim 8.

21. Apparatus for stopping bleeding from a deep narrow wound, comprising:
an elongate hemostatic element formed of a permeable exterior layer containing a volume of a hemostatic agent, shaped for lengthwise insertion into the wound.

22. An improved bandage which includes:
a strip of fabric for winding about a body portion having a wound;
a dressing, formed integrally with said strip of fabric, for placement over the wound; and
a plurality of elongate pockets formed over said dressing, each said pocket adapted to contain a single, discrete, topical agent-bearing element formed of a permeable exterior layer containing a volume of a topical agent for treating a bleeding wound.

23. An improved bandage according to claim 22, wherein said topical agent is a hemostatic agent.
